(19) United States
(12) Patent Application Publication
(10) Pub. No.: US 2013/0175355 A1
(43) Pub. Date: Jul. 11, 2013

(54) ODOR REDUCTION DEVICE

(71) Applicants: Robert William Lackey, Hickory, NC (US); David James McNamara, Huntersville, NC (US); Charles Beach Templeton, Matthews, NC (US); Jeri Lynn Lemke, Hickory, NC (US)

(72) Inventors: Robert William Lackey, Hickory, NC (US); David James McNamara, Huntersville, NC (US); Charles Beach Templeton, Matthews, NC (US); Jeri Lynn Lemke, Hickory, NC (US)

(73) Assignee: PROTECT PLUS AIR, LLC, Hickory, NC (US)

(21) Appl. No.: 13/544,416

(22) Filed: Jul. 9, 2012

Related U.S. Application Data
(60) Provisional application No. 61/505,674, filed on Jul. 8, 2011.

(51) Int. Cl. A61L 9/12

(52) U.S. Cl. CPC A61L 9/12
USPC 239/57

(57) ABSTRACT

The present invention provides methods and systems for an odor reduction device that includes a bottom portion having a base with first and second pairs of spaced-apart, opposed sidewalls projecting from the base to collectively form a cavity to define an upper edge of the device, a top portion that is selectively secured to the bottom portion, and at least one opening in the base of the bottom portion and at least one opening in the top portion that allows air to flow through the device.

### CROSS REFERENCE TO RELATED PATENT APPLICATION

The current application claims the benefit of the earlier priority filing date of the provisional application Ser. No. 61/505,674, that was filed on Jul. 8, 2011.

### FIELD OF THE INVENTION

The present invention relates generally to an odor reduction device and more generally relates to am odor reduction device that contains a filter media that reduces odor disposed within a housing, wherein the housing may be engaged to a filter, HVAC ductwork, register grill, or vent. An optional scent media may also be disposed within the housing.

### BACKGROUND OF THE INVENTION

Many homeowners routinely utilize a spray to neutralize odors or an apparatus to provide a scented fragrance into the habitable space of their homes. The sprays to neutralize odors are limited to a short amount of time and for a limited space. The spray is not continuous and limited in area. For a scented fragrance, candles were typically used to transmit the scent into the air, but today, a number of devices are marketed heavily to provide scented air. However, these devices only provide the scent to the immediate area and for a limited amount of time, such as an hour with a candle. There is a need to provide an odor reducer and/or a scent to a home that has a prolonged life and can be used throughout the residence. It is an object of the present invention to create a device that may be attached to either an air filter or HVAC ductwork for reducing odor and infusing the air passing through the filter or ductwork with a scent.

### BRIEF SUMMARY OF THE INVENTION

According to an embodiment of the present invention, the present invention is an odor reduction device that includes a bottom portion having a base with first and second pairs of spaced-apart, opposed sidewalls projecting from the base to collectively form a cavity to define an upper edge of the device, a top portion that is selectively secured to the bottom portion, and at least one opening in the base of the bottom portion and at least one opening in the top portion that allows air to flow through the device.

According to another embodiment of the present invention, an odor reduction device that includes a plurality of openings in the base of the bottom portion for allowing air to flow through the device.

According to yet another embodiment of the present invention, an odor reduction device that includes a plurality of openings in the top portion for allowing air to flow through the device.

According to yet another embodiment of the present invention, an odor reduction device that includes a rim extending from the upper edge that extends downwardly and outwardly from the upper edge.

According to yet another embodiment of the present invention, an odor reduction device that includes at least one tab that is resiliently flexible for allowing the device to be engaged to an air filter.

According to yet another embodiment of the present invention, an odor reduction device that includes an odor reduction filter disposed within the cavity.

According to yet another embodiment of the present invention, an odor reduction device that includes a scent media that is disposed within the cavity.

According to yet another embodiment of the present invention, an odor reduction device that includes a front slot disposed on the device for receiving a latch disposed on the top portion, wherein the top portion is selectively engaged to the bottom portion.

According to yet another embodiment of the present invention, an odor reduction device that includes a bottom portion having a base with first and second pairs of spaced-apart, opposed sidewalls projecting from the base to collectively form a cavity to define an upper edge of the device. A top portion that is selectively secured to the bottom portion, and a latch disposed on the top portion for selectively engaging the top portion to the bottom portion. A plurality of openings in the base of the bottom portion and a plurality of openings in the top portion that allows air to flow through the device.

According to yet another embodiment of the present invention, an odor reduction device that includes a top portion that has a front side, a back side, a left side, and a right side and a pair of flanges extend outwardly from the back side for selectively securing the top portion to the bottom portion and a latch is disposed on the front side of the top portion.

According to yet another embodiment of the present invention, an odor reduction device that includes at least one resiliently flexible tab containing a bore for receiving an attachment means for attaching the device to a structure and in the stream of air flow.

According to yet another embodiment of the present invention, an odor reduction device that includes a pair of rear slots within the bottom portion for receiving a pair of flanges disposed on the top portion, wherein the pair of flanges are inserted into the pair of rear slots such that the pair of flanges may pivot within the pair rear slots and are easily removed from the pair of rear slots, allowing the top portion to be selective secured to the bottom portion.

According to yet another embodiment of the present invention, an odor reduction device that includes a bottom portion having a base with first and second pairs of spaced-apart, opposed sidewalls projecting from the base to collectively form a cavity to define an upper edge of the device, and at least one rear slot disposed on a sidewall and a front slot disposed on a sidewall. A top portion having at least one rear flange extending outwardly from the top portion for insertion into the at least one rear slot and a latch disposed on the top portion for insertion into the front slot, wherein the top portion is selectively secured to the bottom portion. A filter media is positioned within the cavity, and a plurality of openings in the base of the bottom portion and a plurality of openings in the top portion that allows air to flow through the device.

According to yet another embodiment of the present invention, an odor reduction device that includes a base that having a rib positioned on a sidewall for positioning the filter media over the plurality of openings in the base.

According to yet another embodiment of the present invention, an odor reduction device that includes a rim that extends outwardly from the upper edge and contains at least one resiliently flexible tab positioned on the rim for engagement to an air filter.

According to yet another embodiment of the present invention, an odor reduction device includes knobs disposed on the bottom portion of the device for gripping an air filter.

According to yet another embodiment of the present invention, an odor reduction device includes a shelf positioned within the front slot and at least one finger disposed on the latch, wherein the at least one finger is engaged to the shelf.

According to yet another embodiment of the present invention, an odor reduction device that includes a filter media that has two layers of non-woven media that is impregnated with at least one odor reduction additive.

### BRIEF DESCRIPTION OF THE DRAWINGS

The present invention is illustrated and described herein with reference to the various drawings, in which like reference numbers denote like method steps and/or system components, respectively, and in which:

FIG. 1 is a perspective view of the odor reduction device;

FIG. 2 is an exploded view of the odor reduction device with the odor media and scent media;

FIG. 3 is a perspective view of the odor reduction device engaged to an air filter;

FIG. 4 is a perspective rear view of the odor reduction device;

FIG. 5 is a perspective front view of the bottom portion of the odor reduction device; and

FIG. 6 is a perspective view of the tab on the top portion of the odor reduction device.

### DETAILED DESCRIPTION OF THE INVENTION

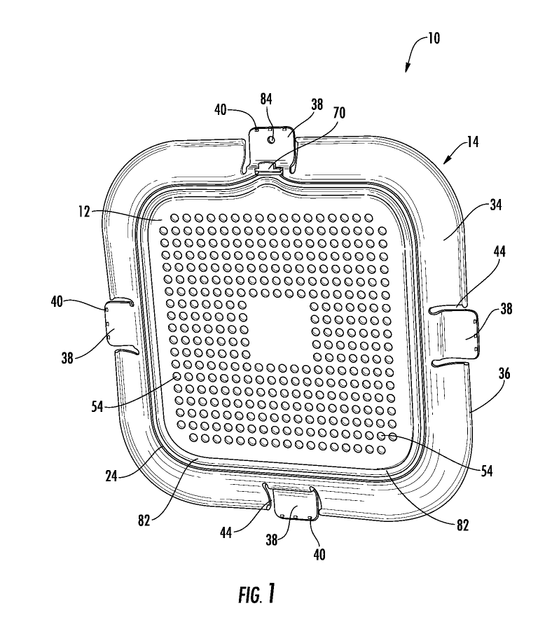

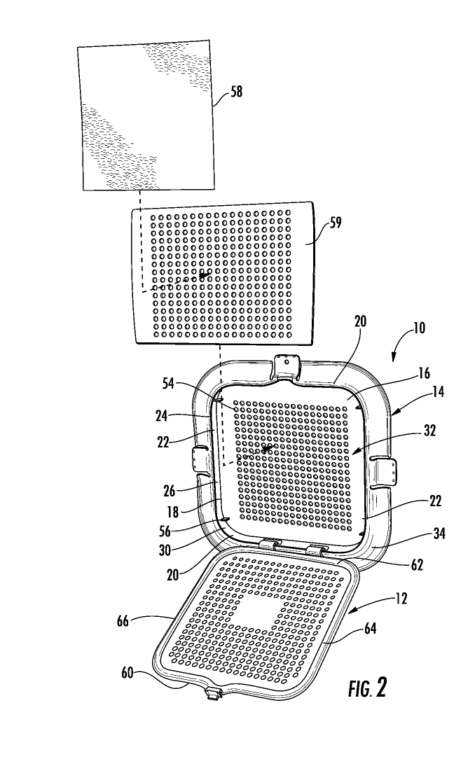

Referring now specifically to the drawings, an odor reduction device is illustrated in FIGS. 1-4 and is shown generally at reference numeral 10. The device 10 includes a top portion 12 and a bottom portion 14. The bottom portion 14 includes a base 16 that extends to an outer edge 18. First and second pairs of opposed sidewalls 20, 22 extend from the outer edge 18 to an upper edge 24. The side walls 20, 22 each have inner and outer sidewall surfaces 26, 28. As shown in FIG. 2, each of the second pair of sidewalls 22 interconnects the first pair of sidewalls 20 to define corners 30. The intersection of the second pair of sidewalls 22 and first pair of sidewalls 20 also defines a cavity 32 therein. A rim 34 extends outwardly from the outer sidewall surfaces 28 and the upper edge 24.

The rim 34 extends outwardly from the outer sidewall surfaces 28 and the upper edge 24 and downwardly. In other words, the rim 34 extends outwardly and downwardly with respect to the upper edge 24. The rim 34 includes a first end and a second end, wherein the first end of the rim 34 is disposed near the upper edge 24 and the second end is opposite the first end. A lip 36 extends substantially perpendicularly upward from the second end of the rim 34.

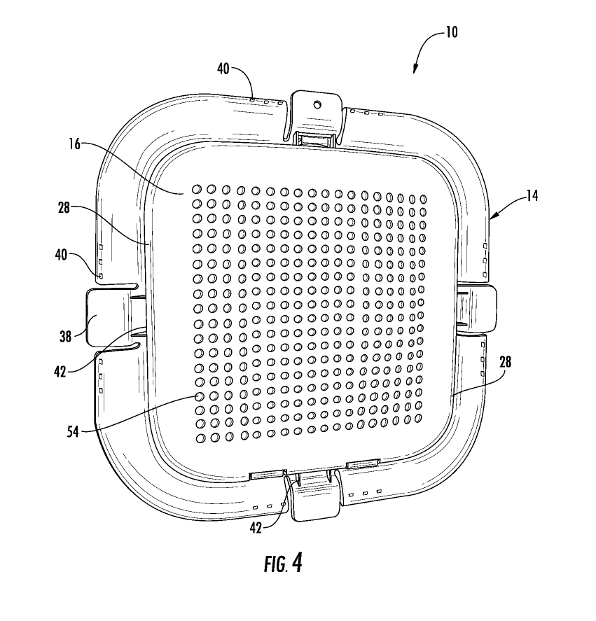

As illustrated in FIG. 1, at least one tab 38 is disposed within the rim 34. The tab 38 has an upper portion, a lower portion, a front side, and a back side. The tab 38 has an upper portion disposed in close proximity to the first end of the rim 34 and upper edge 24. The tab 38 is angled downward from the upper portion to a substantially planer lower portion. At least one knob 40 is disposed on the front side of the lower portion. The tab 38 is resiliently flexible and flexes when a small amount of pressure is exerted on the tab 38. A reinforcement member 42 is positioned on the back side of the tab 38 to add rigidity and stability. As shown in FIG. 4, two reinforcement members 42 are disposed on the back side of the tab 38, but any number of reinforcement members 42 may be utilized. The reinforcement members 42 are disposed between the back side of the tab 38 and the outer sidewall surface 28. The sides of the tab 38 are spaced-apart from the rim 34, thus allowing the tab 38 to flex or move in the forward and backward direction while still being engaged to the rim 34. Preferably, a slit 44 is positioned between the tab 38 and rim 34, allowing the tab 38 to flex or move unobstructed by the rim 34.

As illustrated in FIG. 1, at least one knob 40 is disposed on the back side of the tab 38. The knob 40 is disposed near the lower portion of the back side of the rim 34. As illustrated, a plurality of knobs 40 are disposed on the back side of the rim 34. The knobs 40 are positioned on either side of the tab 38, and an identical number of knobs 40 are positioned on either side of the tab 38 and spaced apart from one another.

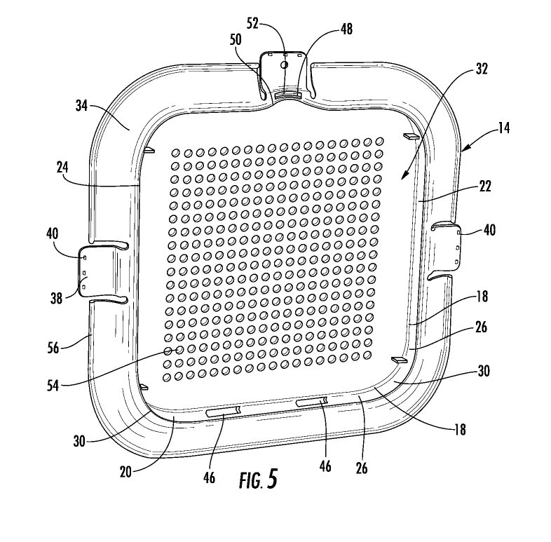

At least one rear slot 46 is positioned on one of the opposed sidewalls 20, 22 and extends from the inner sidewall surface 26 to the outer sidewall surface 28. The bottom portion of the rear slot 46 is preferably adjacent the outer edge 18 of the base 16. The upper portion of the rear slot 46 is positioned along the length of the sidewalls 20, 22 on which it is positioned. As illustrated in FIG. 5, the device 10 preferably contains two spaced-apart rear slots 46, containing the above described features. The two spaced-apart rear slots 46 are positioned on one of the opposed sidewalls 20, 22 and extend from the inner sidewall surface 26 to the outer sidewall surface 28. The bottom portion of the rear slot 46 is preferably adjacent the outer edge 18 of the base 16. The upper portion of the rear slot 46 is positioned along the length of the sidewalls 20, 22 on which it is positioned. The two rear slots 46 are spaced on either side of the tab 38 that is positioned on the rim 34 external to the two rear slots 46.

One of the opposed sidewalls 20, 22 contains a front slot 48 disposed on the rim 34. Preferably, this front slot 48 is positioned on the sidewall opposite the sidewall containing the at least one rear slot 46. For example, the at least one rear slot 46 is positioned on sidewall 20, and the front slot 48 is positioned on the opposed sidewall 20. Alternatively, the at least one front slot 46 is positioned on sidewall 22, and the front slot 48 is positioned on the opposed sidewall 22. The front slot 48 is positioned at the intersection of the upper edge 24 and the rim 34. The area where the front slot 48 is located contains a depression 50 formed within the upper edge 24, rim 34, and the sidewall 20, 22 in which the front slot 48 is located. In other words, the front slot 48 is positioned lower than the upper edge 24 surrounding the device 10. Based upon the gradual slope of the sides of the depression 50, this may result in a front slot 48 that is curved within the bottom of the depression 50.

The front slot 48 contains a front part and a back part. The front part contains a top surface and a bottom surface, and similarly, the back part contains a top surface and a bottom surface. The bottom surface of the front part of the front slot 48 contains a shelf 52 that is integrally formed within the device 10. As illustrated in FIG. 5, the shelf 52 spans substantially the length of the front slot 48. However, the shelf 52 may be of a length and width that is suitable for its intended purposes.

The base 16 contains at least one opening 54, and as illustrated in FIG. 5, the device 10 contains a plurality of openings 54 that are spaced-apart and equal distance from each other. Additionally, the openings 54 are of the same diameter and are arranged having a symmetrical pattern. In an alternative embodiment, the openings 54 may have any shape, size, or diameter and arranged in any pattern or arrangement so as to accomplish the plurality of openings 54 intended function. Alternatively, the base 16 of the device 10 may contain a single opening 54, have two openings 54, or contain a plurality of openings 54.

At least one rib 56 is positioned within the base 16 for properly aligning a filter media 58 and/or scent media 59 that is placed within the cavity 32. The purpose of the at least one rib 56 is to position the filter media 58 and/or scent media 59 within the central portion of the cavity 32 so that the filter media 58 and/or scent media 59 is positioned adjacent the openings 54. As illustrated in FIG. 2, two spaced-apart ribs 56 are positioned on sidewall 20 and two spaced-apart ribs 56 are positioned on the opposed sidewall 20. It will be understood, that the two spaced-apart ribs 56 may be positioned on sidewall 22 and two spaced-apart ribs 56 may be positioned on the opposed sidewall 22. The bottom of the rib 56 is positioned along the base 16 and one edge of the rib 56 is engaged to the respective sidewall 20, 22 while the opposed edge of the rib 56 is designed to contact the filter media and/or scent media 58 for correct positioning.

The top portion 12 of the device 10 is designed to fit within the cavity 32 and is selectively secured to the bottom portion 14. The top portion 12 contains a front side 60, back side 62, left side 64, and right side 66. The back side 62 of the top portion 12 contains two flanges 68 that extend downwardly from the top portion 12. Preferably, the flanges 68 extend downwardly from the top portion 12 and then curve outward and away from the top portion 12. The curvature of the flanges 68 allow the flanges 68 to be inserted into the rear slots 46 and allows the top portion 12 to be raised upwards without disengaging the flanges 68 from the rear slots 46. The shape of the flanges 68 allow the flanges 68 to be easily inserted into the rear slots 46, allow the flanges 68 to pivot in the upward and downward direction within the rear slots 46, and allow the flanges 68 to easily be removed from the rear slots 46.

The top portion 12 contains at least one opening 54, and as illustrated in FIGS. 1-3, the top portion contains a plurality of openings 54 that are spaced-apart and equal distance from each other. Additionally, the openings 54 are of the same diameter and are arranged having a symmetrical pattern. In an alternative embodiment, the openings 54 may have any shape, size, or diameter and arranged in any pattern or arrangement so as to accomplish the plurality of openings 54 intended function. Alternatively, the top portion 12 of the device 10 may contain a single opening 54, have two openings 54, or contain a plurality of openings 54.

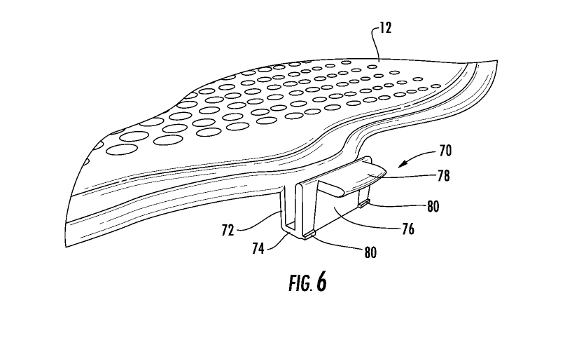

The front side 60 of the top portion 12 contains a frustoconical center section wherein a latch 70 is disposed thereon. The latch 70 is designed to be received within the front slot 48. Any latch 70 may be utilized that would allow the top portion 12 to be selectively secured to the bottom portion 14. As illustrated in FIG. 6, the latch 70 consists of a first vertical portion 72 that is engaged to a horizontal portion 74 that is engaged to the second vertical portion 76. A finger 78 extends from the second vertical portion 76 and allows a user to disengage the latch 70 from the front slot 48. The horizontal portion 74 creates a spaced-apart relationship between the first vertical portion 72 and the second vertical portion 76, which allows the second vertical portion 76 to flex or bend towards the first vertical portion 72. The second vertical portion 76 contains a grip 80 or grips 80 that are designed to engage the shelf 52 of the front slot 48. The grips 80 are engaged to the shelf 52 by friction fit.

When a user desires to raise the top portion 12 of the device 10, the user lifts the finger 78. As pressure is applied to the finger 78 by the user, the second vertical portion 76 bends towards the first vertical portion 72 and also pivots in the upward and downward direction with respect to the edge of the top portion 12. As the second vertical portion 76 bends towards the first vertical portion 72, the grips 80 disengage the shelf 52, allowing the user to pull the top portion 12 upward and separate the top portion 12 from the bottom portion 14. Additionally, as the first vertical portion 72 pivots with respect to the edge of the top portion, the grips 80 also disengage the shelf 52, allowing the user to pull the top portion 12 upward and separate the top portion 12 from the bottom portion 14. A channel 82 circumscribes the outer edge of the top portion 12. The channel 82 is positioned around the plurality of openings 54.

The filter media 58 absorbs odor molecules as they pass through or in close proximity to the filter media 58. The filter media 58 may take any form and the description herein of the filter media 58 is not meant to be limiting. In an exemplary embodiment, the filter media 58 is composed of 2 layers of nonwoven media treated with baking soda and carbon granules. The 2 layers are engaged together, such as with an adhesive. In another exemplary embodiment, the filter media 58 is composed of 5 layers. Three of the layers are a non-woven media that is impregnated with baking soda. The other two layers are composed of a carbon/paper sheet. The layers alternate and form a five layer structure. The filter media 58 is replaceable and may be replaced by removing the top portion 12 or alternatively through a slit in the side of the device 10 allowing the filter media 58 to be inserted and removed easily from the device 10. In an alternative embodiment, the filter media 58 is at least one layer of nonwoven media that is treated with baking soda or carbon granules. Additionally, at least one odor reduction additive that is commonly known to one of ordinary skill in the art may be engaged to the nonwoven media. The odor reduction additive may be impregnated into the nonwoven media, adhered to the nonwoven media with an adhesive, or enclosed inside a pouch.

An optional scent media 59 may be placed adjacent the filter media 58 and opposite the flow of air, so that the air passes through the filter media 58 and then the scent media 59, as illustrated in FIG. 2. Alternatively, the device 10 may contain only the scent media 59 without the filter media 58. The scent media may be in the form of a single or multi-layered chemically treated media or powdered chemicals blended together and sealed in a non-woven media bag. Alternatively, the scent media 59 may be a molded scent device or a housing that contains scented beads. For the ease of communication, the term scented media is used to incorporate chemically treated media, carbon sheets, or powdered chemicals sealed in a non-woven media bag. The scent media 59 is designed to provide 30-45 days worth of odor reduction and/or elimination in an HVAC system that operates 8 hours in a 24 hour time period. The scent media 59 is replaceable and may be replaced by removing the top portion 12 or through a slit in the side of the device 10, allowing the scented media to be inserted and removed easily from the device 10. In an alternative embodiment, the scented media 59 may be composed of a low density polyethylene or high density polyethylene based resin that is blended with an ethylene-vinyl acetate and scented oils that have a concentration of up to 30%.

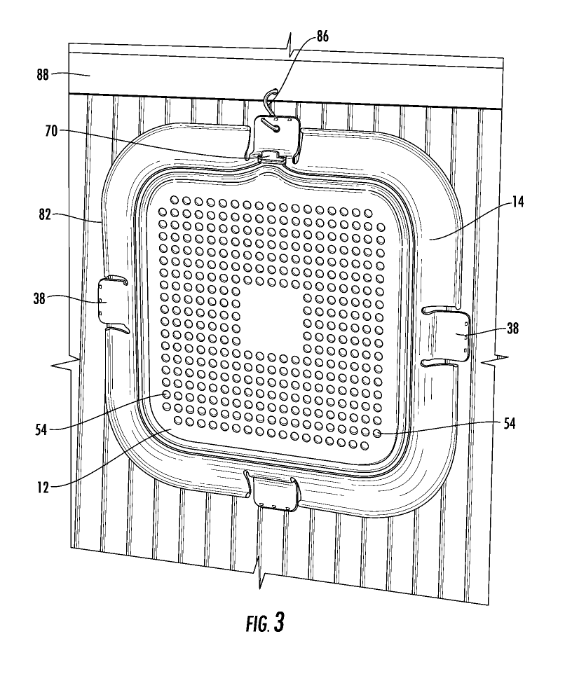

During use, the device 10 is placed within the stream of air so that the air can pass through the openings 54 in the bottom portion 14 and the openings 54 in the top portion 12. The device 10 may be engaged to an air filter, HVAC ductwork, register, grill, vent or the like. The tabs 38 are designed to be engaged to the filter frame of an air filter. The filter frame is placed within the slit 44, allowing the tab 38 to be placed behind the filter frame. The knobs 40 on the tab 38 engage the filter frame, and likewise, the knobs 40 of the rim engage the filter frame. A tab 38 may also contain a bore 84 for receiving a hook 86. As illustrated in FIG. 3, the hook 86 is an s-shaped hook that is has a first end and a second end, whereby the first end of the hook 86 is received within the bore 84 and the second end is engaged to the filter 88, HVAC ductwork, register, grill, vent or the like for positioning the device 10 within the stream of air flow.

Although the present invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention and are intended to be covered by the following claims.

What is claimed is:

1. An odor reduction device, comprising:
a bottom portion having a base with first and second pairs of spaced-apart, opposed sidewalls projecting from the base to collectively form a cavity to define an upper edge of the device;
a top portion that is selectively secured to the bottom portion; and
at least one opening in the base of the bottom portion and at least one opening in the top portion that allows air to flow through the device.

2. The odor reduction device of claim 1, further comprising a plurality of openings in the base for allowing air to flow through the device.

3. The odor reduction device of claim 1, further comprising a plurality of openings in the top portion for allowing air to flow through the device.

4. The odor reduction device of claim 1, further comprising a rib extending from the upper edge that extends downwardly and outwardly from the upper edge.

5. The odor reduction device of claim 1, further comprising at least one tab that is resiliently flexible for allowing the device to be engaged to an air filter.

6. The odor reduction device of claim 1, further comprising a filter media that is disposed within the cavity.

7. The odor reduction device of claim 1, further comprising a scent media disposed within the cavity.

8. The odor reduction device of claim 1, further comprising a front slot disposed on the device for receiving a latch disposed on the top portion, wherein the top portion is selectively engaged to the bottom portion.

9. An odor reduction device, comprising:
a bottom portion having a base with first and second pairs of spaced-apart, opposed sidewalls projecting from the base to collectively form a cavity to define an upper edge of the device;
a top portion that is selectively secured to the bottom portion;
a latch disposed on the top portion for selectively engaging the top portion to the bottom portion; and
a plurality of openings in the base of the bottom portion and a plurality of openings in the top portion that allows air to flow through the device.

10. The odor reduction device of claim 9, wherein the top portion has a front side, a back side, a left side, and a right side and a pair of flanges extending outwardly from the back side for selectively securing the top portion to the bottom portion and a latch disposed on the front side of the top portion.

11. The odor reduction device of claim 9, further comprising a filter media is disposed within the cavity.

12. The odor reduction device of claim 9, further comprising a scent media disposed within the cavity.

13. The odor reduction device of claim 9, further comprising at least one resiliently flexible tab containing a bore for receiving an attachment means for attaching the device to a structure and in the stream of air flow.

14. The odor reduction device of claim 9, further comprising a pair of rear slots within the bottom portion for receiving a pair of flanges disposed on the top portion, wherein the pair of flanges are inserted into the pair of rear slots such that the pair of flanges are easily removed from the pair of rear slots and allow the top portion to be selective secured to the bottom portion.

15. An odor reduction device, comprising:
a bottom portion having a base with first and second pairs of spaced-apart, opposed sidewalls projecting from the base to collectively form a cavity to define an upper edge of the device, and at least one rear slot disposed on a sidewall and a front slot disposed on a sidewall;
a top portion having at least one rear flange extending outwardly from the top portion for insertion into the at least one rear slot and a latch disposed on the top portion for insertion into the front slot, wherein the top portion is selectively secured to the bottom portion; and
a filter media is positioned within the cavity; and
a plurality of openings in the base of the bottom portion and a plurality of openings in the top portion that allows air to flow through the device.

16. The odor reduction device of claim 15, wherein the base contains a rib positioned on a sidewall for positioning the filter media over the plurality of openings in the base.

17. The odor reduction device of claim 15, further comprising a rim extending outwardly from the upper edge and containing at least one resiliently flexibly tab positioned on the rim for engagement to an air filter.

18. The odor reduction device of claim 15, further comprising knobs disposed on the bottom portion of the device for gripping an air filter.

19. The odor reduction device of claim 15, further comprising a shelf positioned within the front slot and at least one finger disposed on the latch, wherein the at least one finger is engaged to the shelf.

20. The odor reduction device of claim 15, wherein the filter media comprises two layers of non-woven media that is impregnated with at least one odor reduction additive.
